(19) United States
(12) Patent Application Publication
(10) Pub. No.: US 2023/0255496 A1
(43) Pub. Date: Aug. 17, 2023

(54) Measurement Device

(71) Applicant: Nippon Telegraph and Telephone Corporation, Tokyo (JP)

(72) Inventors: Yujiro Tanaka, Tokyo (JP); Daichi Matsunaga, Tokyo (JP)

(21) Appl. No.: 18/003,773

(22) PCT Filed: Jul. 13, 2020
(86) PCT No.: PCT/JP2020/027245

§ 371 Date: Dec. 29, 2022

(51) Int. Cl. A61B 5/01; G01K 13/20

(52) U.S. Cl. CPC A61B 5/01; G01K 13/20; A61B 2560/0412; A61B 2562/0271; A61B 2562/04

(57) ABSTRACT

A measurement device includes: a first cover member having a measuring instrument; a second cover member forming an air layer between the first cover member and the second cover member; and a third cover member that transports heat flux from a measurement target outside the first cover member to an upper portion of the second cover member between the first cover member and the second cover member.

### CROSS-REFERENCE TO RELATED APPLICATIONS

This application is a national phase entry of PCT Application No. PCT/JP2020/027245, filed on Jul. 13, 2020, which application is hereby incorporated herein by reference.

### TECHNICAL FIELD

The present invention relates to a measurement device for measuring a core temperature of a measurement target such as a living body.

### BACKGROUND

Conventionally, a technique for non-invasively measuring a core temperature of a living body is known. For example, Patent Literature 1 discloses a technique for estimating a core temperature of a living body assuming a pseudo one-dimensional model in a living body B, a measuring instrument 40 including a temperature sensor and a heat flux sensor, and outdoor air.

In the technique disclosed in Patent Literature 1, a core temperature of a living body is estimated assuming a one-dimensional model of biological heat transfer illustrated in FIG. 10. Tair is the temperature of the outdoor air, Tbody is the core temperature of the living body B, Hsignal is the heat flux flowing into the sensor of the measuring instrument 40, Rbody is the thermal resistance of the living body B, Rair is the thermal resistance when the heat flux Hsignal moves to the outdoor air, Tskin is the temperature of the contact point between the temperature sensor arranged on the skin SK and the skin SK of the living body B, and Tt is the temperature of the arrangement position of the upper temperature sensor.

In Patent Literature 1, a core temperature of a living body is estimated from the following relational expression (i).

Core temperature (Tbody)=Temperature (Tskin) of contact point between temperature sensor and skin+Coefficient of proportionality (Rsensor)×Heat flux (Hsignal) flowing into temperature sensor (1)

The coefficient of proportionality (Rsensor) can be generally obtained by giving a rectal temperature or an eardrum temperature measured using a sensor such as another temperature sensor as a core temperature (Tbody), and thus the core temperature of the living body can be estimated by measuring a heat flux (Hsignal) flowing into the temperature sensor.

### CITATION LIST

### Patent Literature

Patent Literature 1: JP 2020-003291 A

### SUMMARY

### Technical Problem

However, in a case where a one-dimensional model is assumed as a heat transfer model of a living body as in Patent Literature 1, when heat flows into the sensor from outdoor air due to generation of wind or the like, as illustrated in FIG. 11, a part of the heat flux Hsignal that should originally flow into the sensor deviates from the sensor.

FIG. 12 illustrates the heat equivalent circuit. Rbody is a thermal resistance of a living body, RLeak is a biological thermal resistance when heat flows to outdoor air due to wind or the like and moves while being deviated from a flow of heat originally passing therethrough, and a leaking heat flux is HLeak. Rair and R′air are thermal resistances when Hsignal and HLeak move to the outdoor air, respectively. When the thermal resistance between the sensor and the outdoor air is changed by the wind and the heat flux HLeak deviating from the sensor and leaking is generated, the heat flux Hsignal to be originally measured is decreased by that amount and becomes H′signal. Here, the influence of HLeak on Hsignal is evaluated by a ratio Leak Ratio of HLeak to Hsignal. The Leak Ratio is represented by |HLeak|/Hsignal.

Therefore, when wind or the like is generated, the Leak Ratio increases, and the above-described one-dimensional model is no longer established for the Hsignal, and in the core temperature measurement technique of the related art, there is a problem that an erroneous core temperature is measured when wind or the like is generated around the sensor.

Embodiments of the present invention have been made to solve the above-described problems, and an object of embodiments of the present invention is to provide a measurement device capable of accurately measuring a core temperature by suppressing a change in thermal resistance between a sensor and outdoor air even when wind or the like is generated around the sensor.

### Solution to Problem

In order to solve the above-described problems, there is provided a measurement device according to embodiments of the present invention including: a measuring instrument configured to measure a heat flux transported from a measurement target; a first member having a hollow structure and including the measuring instrument therein; a second member having a hollow structure and covering the first member to form an air layer between the first member and the second member; and a third member that is disposed between the first member and the second member and transports the heat flux from the measurement target outside the first member to an upper portion of the second member.

### Advantageous Effects of Embodiments of the Invention

According to embodiments of the present invention, since the first member having the measuring instrument, and the second member forming the air layer between the first member and the second member, are provided, and the third member that transports the heat flux from the measurement target outside the first member to the upper portion of the second member is further provided between the first member and the second member, it is possible to provide a measurement device capable of suppressing the change in the thermal resistance between the sensor and the outdoor air and accurately measuring the core temperature even when wind is generated around the sensor.

### BRIEF DESCRIPTION OF THE DRAWINGS

FIG. 1 is an example of a cross-sectional view of a measurement device according to an embodiment of the present invention.

FIG. 2 is another example of a cross-sectional view of the measurement device according to the embodiment of the present invention.

FIG. 3 is a view illustrating an example of second and third members of the measurement device according to the embodiment of the present invention.

FIG. 4 is a view illustrating an example of the third member of the measurement device according to the embodiment of the present invention.

FIG. 5 is a view illustrating an example of a cross-sectional view of the third member of the measurement device according to the embodiment of the present invention.

FIG. 6 is a view illustrating a temperature field and a heat flux in the vicinity of the measurement device according to the embodiment of the present invention.

FIG. 7 is a view illustrating a heat equivalent circuit of FIG. 6.

FIG. 8 is a measurement result of a measurement error of a core temperature according to the embodiment of the present invention.

FIG. 9 is an example of a block diagram of a measurement device according to the embodiment of the present invention.

FIG. 10 is a heat equivalent circuit for estimating the core temperature by the heat flux.

FIG. 11 is a view for describing a leak heat flux at the time of estimating the core temperature by heat flux.

FIG. 12 is a heat equivalent circuit view of a case in which a leakage heat flux is generated.

### DETAILED DESCRIPTION OF ILLUSTRATIVE EMBODIMENTS

Hereinafter, a preferred embodiment of the present invention will be described. In the following embodiment, the measurement target is a living body, and the measurement surface on which the measurement device is disposed is a surface of the skin of a living body that is the measurement target.

<Summary of Embodiments of Present Invention>

The measurement device of embodiments of the present invention includes a first member having a hollow structure and including a measuring instrument for measuring a heat flux therein, and a second member having a hollow structure and forming an air layer between the second member and the first member, and further includes a third member that transports a heat flux from a measurement target outside the first member to an upper portion of the second member between the first member and the second member.

In the measurement device of embodiments of the present invention, the third member that transports the heat flux from the measurement target to the upper portion of the second member is provided in addition to the first member having the measuring instrument that measures the heat flux and the second member forming the air layer between the first member and the second member, and accordingly, it is possible to increase the temperature of the upper portion of the measuring instrument. Therefore, even when wind is generated around the measurement device, it is possible to suppress the change in the thermal resistance between the measuring instrument and the outdoor air, suppress the leak heat flux that causes the measurement error of the core temperature, and reduce Leak Ratio. Hereinafter, a specific configuration of the measurement device of the present embodiment will be described.

<Configuration of Measurement Device>

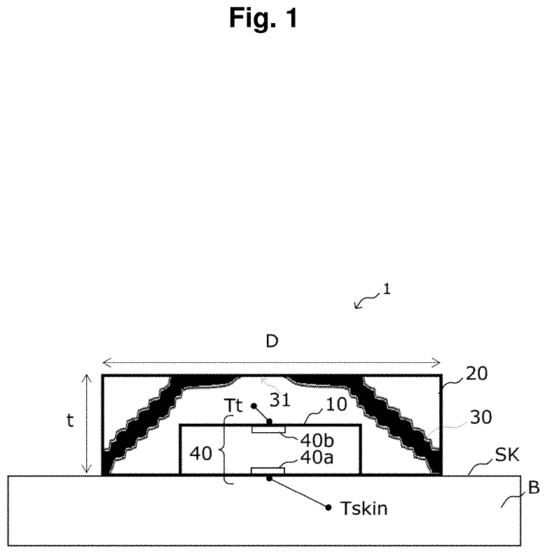

FIG. 1 illustrates an example of a cross-sectional view of the measurement device according to an embodiment of the present invention. FIG. 1 illustrates a configuration example of a first member including a measuring instrument 40 therein, a second member covering the first member, and a third member disposed in a space between the first member and the second member. The measuring instrument 40 disposed inside the first member includes a sensor that measures the heat flux transported from a living body B. Furthermore, although not illustrated in this drawing, the measurement device 1 includes an arithmetic circuit or the like for estimating the core temperature of the living body B in addition to the configuration of the measurement device 1 of FIG. 1.

The measurement device 1 of FIG. 1 includes a first member 10 having a hollow structure that holds the measuring instrument 40 therein, a second member 20 having a hollow structure that covers the first member 10 and forms an air layer between the first member 10 and the second member 20, and a third member 30 having a truncated shape and having a hollow shell structure that is disposed in a space between the second member 20 and the first member 10.

In the configuration example of FIG. 1, the truncated upper surface portion of the third member 30 is in contact with the upper surface portion of the second member 20 from the inside of the second member 20. The truncated third member 30 has a hole portion 31 penetrating the third member 30 on the upper surface portion thereof. By bringing the upper surface portion of the third member 30 into contact with the upper surface portion of the second member 20, the heat flux from the living body B outside the first member 10 is transported to the upper surface portion of the second member 20.

The measuring instrument 40 disposed inside the first member 10 includes a temperature sensor 40a (first temperature sensor) configured to measure the temperature of skin SK as a measurement surface, and a temperature sensor 40b (second temperature sensor) disposed at a position immediately above the temperature sensor 40a to face the temperature sensor 40a. In the configuration example of FIG. 1, the heat flux is measured using the temperature difference between the temperature Tskin measured by the temperature sensor 40a and the temperature Tt measured by the temperature sensor 40b.

The first member 10 has a hollow structure, and the inside thereof is filled with air. The second member 20 is desirably filled with a material having high thermal resistance, and a cavity such as air can be used.

As the first member 10 and the second member 20, a material (approximately 0.1 mm) capable of reducing the thermal resistance and the thickness is desirable, and polyethylene terephthalate (PET) or the like can be used. As a material constituting the truncated third member 30 having a hollow shell structure, a material having high thermal conductivity is desirable in order to efficiently transport heat flux. For example, the third member 30 can be configured using a thin film such as aluminum or the like.

The first member 10 is disposed on the skin SK of the living body B as a measurement surface. The first member 10 has a hollow structure formed of a thin film, and can have, for example, a cylindrical outer shape. The second member 20 is disposed on the skin SK of the living body B as a measurement surface to cover the first member 10, and forms an air layer between the first member 10 and the second member 20. Similarly to the first member 10, the second member 20 has a hollow structure formed of a thin film, and can have a cylindrical outer shape. Furthermore, the outer shapes of the first member 10 and the second member 20 are not limited to the cylindrical shape, and may be, for example, a rectangular parallelepiped shape having a hollow structure.

The diameters D of the cylindrical shape of the first member 10 and the cylindrical shape of the second member 20 can be, for example, 20 mm and 30 mm, respectively. A height t of the second member 20 based on the skin SK that is a measurement surface can be, for example, approximately 6 mm. A height t of the first member 10 based on the skin SK that is a measurement surface can be, for example, approximately 3 mm.

In this manner, the air layer formed by the first member 10 and the air layer between the first member 10 and the second member 20 on the outer side thereof are formed, and the air inside each of the first member 10 and the second member 20 is configured not to move.

Furthermore, the third member 30 is disposed between the first member and the second member, the upper surface portion thereof is brought into contact with the upper surface portion of the second member 20, and accordingly, the heat flux from the living body B is transported to the upper portion of the second member outside the first member. In the example of FIG. 1, since the truncated third member 30 includes the hole portion 31 penetrating the third member 30 on the upper surface portion thereof, the truncated third member 30 is in contact with the upper surface portion of the second member 20 at the peripheral part of the hole portion 31 of the upper surface portion.

<Configuration of Sensor in Measuring Instrument>

The temperature sensor 40a is disposed on an inner surface of a bottom surface portion where the cylindrical first member 10 is in contact with the skin SK that is a measurement surface. On the inner surface of the upper surface portion of the first member 10, the temperature sensor 40b is disposed at a position immediately above the temperature sensor 40a to face the temperature sensor 40a. In the configuration example of FIG. 1, the heat flux H′signal is measured by the temperature difference between the pair of temperature sensors 40a and 40b.

In FIG. 1, the temperature sensor 40a is disposed in contact with the surface of the skin SK of the living body B, which is a measurement surface, and measures the temperature Tskin (temperature of the measurement surface), which is the temperature of the contact point with the living body B. The temperature sensor 40b measures the temperature Tt at the position where the inner surface of the first member 10 is disposed. As the temperature sensors 40a and 40b, for example, a thermistor, a thermocouple, a platinum resistor, an IC temperature sensor, or the like can be used.

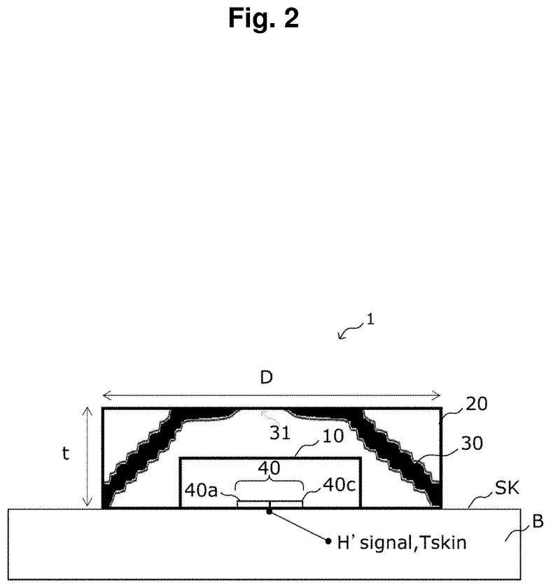

In the configuration example of FIG. 1, the heat flux H′signal is measured by the pair of temperature sensors 40a and 40b. However, as illustrated in FIG. 2, the temperature Tskin of the measurement surface may be measured by the temperature sensor 40a, and the heat flux H′signal may be measured by the heat flux sensor 40c.

In FIG. 2, the heat flux sensor 40c is a sensor that detects heat transfer per unit time and per unit area, and measures a heat flux H′signal [W/m²] flowing from the living body B into the heat flux sensor 40c. As the heat flux sensor 40c, for example, a laminated structure, a planar development type operation type thermopile, or the like can be used. The heat flux sensor 40c is disposed in contact with the surface of the skin SK of the living body B, which is a measurement surface.

In FIG. 2, similarly to FIG. 1, the temperature sensor 40a is disposed in contact with the skin SK that is a measurement surface, and measures the skin temperature Tskin that is the temperature of the contact point with the living body B. The temperature sensor 40a is disposed adjacent to the heat flux sensor 40c along the measurement surface.

<Configuration Example of Third Member>

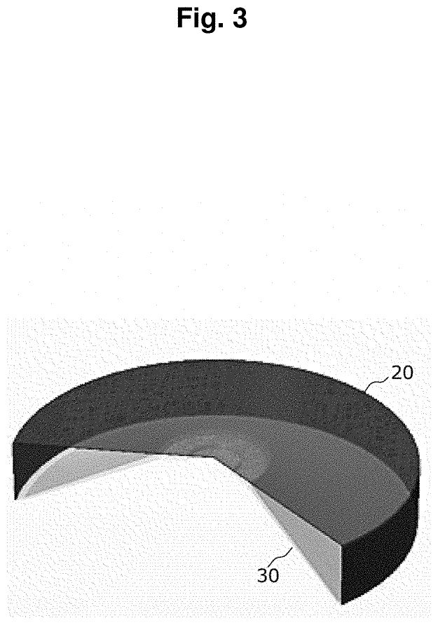

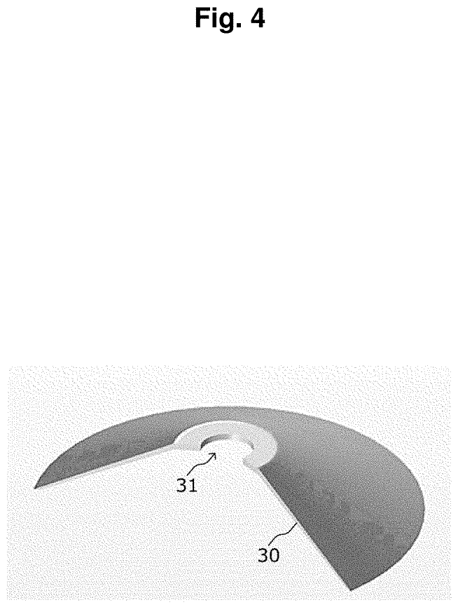

FIGS. 3 and 4 illustrate a configuration example of the third member 30. In FIGS. 3 and 4, the cylindrical second member 20 is disposed to cover the truncated third member 3o, and an upper surface portion of the truncated third member 30 is configured to be in contact with an upper surface portion of the cylindrical second member 20. The truncated third member 30 has a circular hole portion 31 penetrating the third member 30 on the upper surface portion thereof.

The third member 30 is a member that is disposed in the space between the first member 10 and the second member 20, transports the heat flux from the measurement target to the upper surface portion of the second member outside the first member to increase the temperature of the upper surface portion of the second member, that is, the temperature of the upper portion of the measuring instrument 4o, and thereby functions to suppress the leakage heat flux HLeak and reduce the Leak Ratio. As the configuration of the third member 30, configurations of various shapes can be adopted as long as the shape can exhibit this function.

For example, in the case of being disposed between the first member and the second member having a cylindrical shape, the third member can be configured to have a conical shape. By forming the third member in a conical shape, it is possible to transport the heat flux from the measurement target to the upper surface portion of the second member outside the first member without affecting the heat flux flowing into the measuring instrument 40. It can also be configured to have a truncated shape as shown in FIGS. 3 and 4.

Further, the configuration of the third member 30 is not limited to the conical shape or the truncated shape, and other cone shapes can be adopted. For example, when the second member 20 has a rectangular parallelepiped shape, the third member 30 can accordingly have a pyramid shape or a truncated pyramid shape. By forming the third member in a frustum shape, a larger amount of heat flux can be transported to the second member without affecting the heat flux flowing into the measuring instrument 40, and the effect of temperature rise can be enhanced.

As illustrated in FIGS. 1 to 4, the truncated third member 30 may have a circular hole portion 31 penetrating the third member 30 on the upper surface portion thereof. By appropriately adjusting the size of the circular hole portion 31, it is possible to adjust the depth to be measured in a case of measuring the core temperature of the living body B.

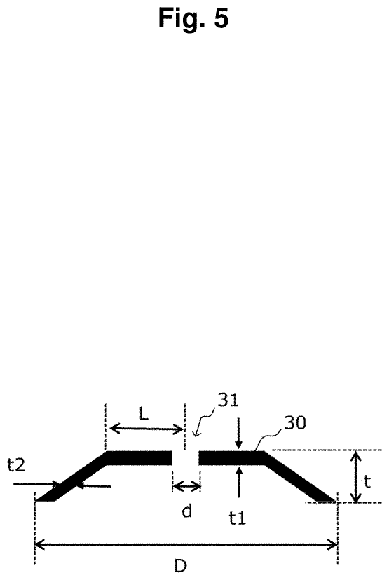

FIG. 5 is an example of a cross-sectional view of the truncated third member 3o having the hole portion 31 in the upper surface portion. As an example of the size of the third member 30 in the present embodiment, in a case where the diameter D of the second member 20 is 30 mm and the height t is 5 mm, the radius L of the upper surface portion is 3 mm to 6 mm, and the diameter d of the hole portion 31 is approximately 1 mm to 3 mm. The thicknesses t1 and t2 of the third member 30 are desirably, for example, approximately 0.3 mm to 1 mm.

<Temperature Field and Heat Flux in Present Embodiment>

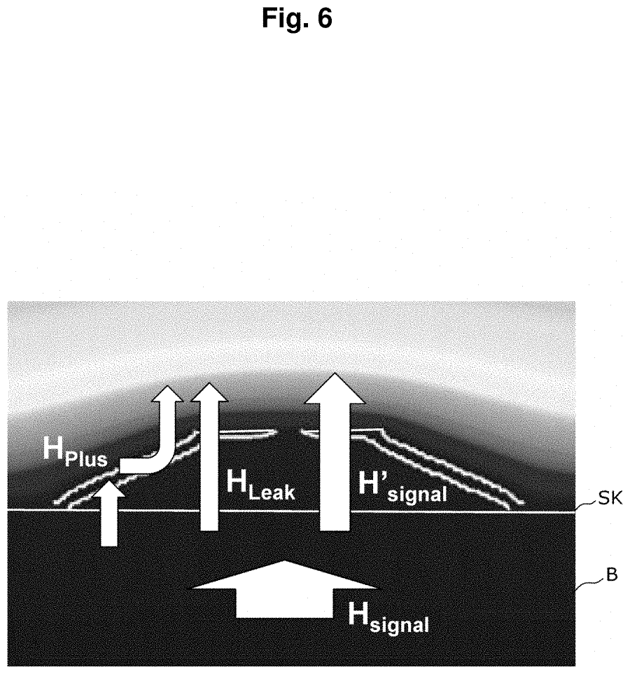

FIG. 6 is a view illustrating a temperature field and a heat flux in the vicinity of the measurement device. A heat flux Hplus is a heat flux transported from the living body B to the vicinity of the central portion of the upper portion of the second member 20 outside the first member 10 via the truncated third member 30.

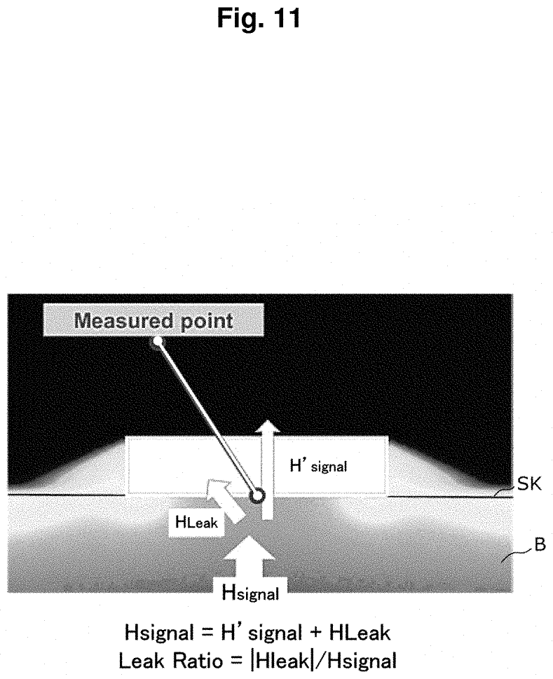

In FIG. 6, Hsignal is a heat flux transported from the core of the living body B, H′signal is a heat flux separated from the Hsignal and flowing into the central temperature sensor, and HLeak is a leak heat flux separated from the Hsignal, deviated from the measuring instrument 40, and escaping to the outside. Similarly to FIG. 11, in this case, the ratio Leak Ratio of HLeak to Hsignal is represented by |HLeak|/Hsignal.

<Heat Equivalent Circuit of Present Embodiment>

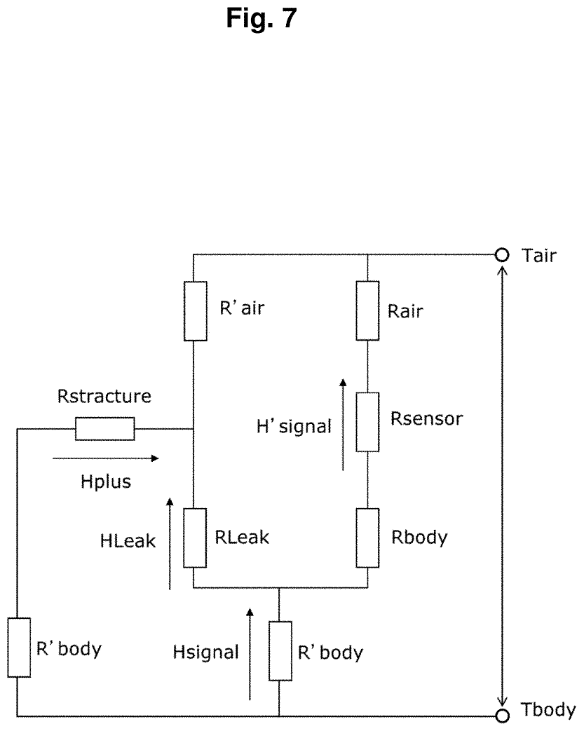

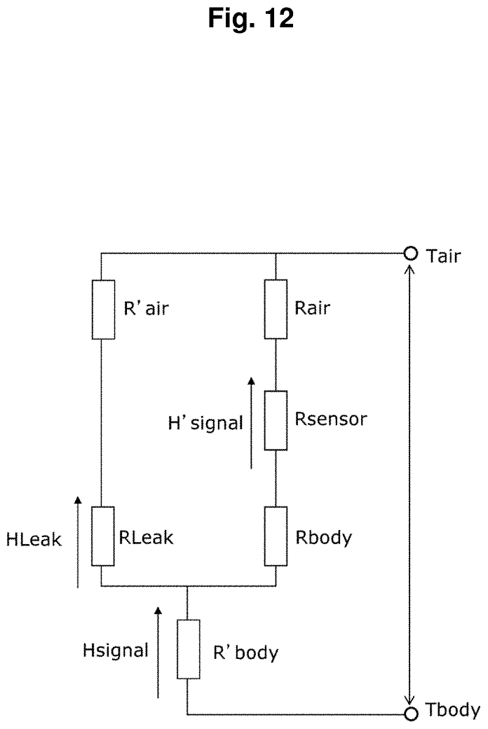

The heat equivalent circuit of FIG. 6 is shown in FIG. 7. Rstructure is a thermal resistance of the truncated third member 3o, R′body is a thermal resistance when heat is transported from the core to the truncated third member 3o, and is a thermal resistance when HLeak moves to the outdoor air as described in FIG. 12. Rair and R′air are thermal resistance when heat is transported to the outdoor air through the measuring instrument 40, and thermal resistance when heat is deviated from the measuring instrument 40 and transported to the outdoor air, respectively.

Here, when the truncated third member 30 is sufficiently large, the end portion of the bottom surface of the truncated third member 30 is disposed at a position sufficiently away from the measuring instrument 40, and thus, the heat flux from the living body B is collected by the third member 30 on the first outer side and transported to the upper surface portion of the second member 20.

The heat flux Hplus collected and transported by the truncated third member 3o increases the temperature of the upper surface portion of the second member 20 without affecting the Hsignal, and as a result, the temperature outside the measuring instrument 40 can be increased. In the heat equivalent circuit of FIG. 7, the heat flux Hplus flows into R′air, and accordingly, the temperature outside the measuring instrument increases, and it is possible to suppress the leakage heat flux HLeak that causes an error and to cause an effect of reducing the Leak Ratio.

The truncated third member 30 is covered with the second member 20, and the distance to the outdoor air decreases toward the vicinity of the central portion where the measuring instrument 40 is disposed, and becomes substantially 0 in the vicinity of the central portion where the measuring instrument 40 is disposed. Accordingly, the effect of suppressing the inflow of heat from the outdoor air to the sensor increases toward the vicinity of the central portion, and the effect of reducing the highest Leak Ratio can be obtained in the vicinity of the central portion where the measuring instrument 40 is disposed. As a result, it is possible to reduce the difference between the heat flux H′signal measured by the temperature sensor or the heat flux sensor and the Hsignal to be originally measured, and to reduce the measurement error.

<Comparison Result of Measurement Error>

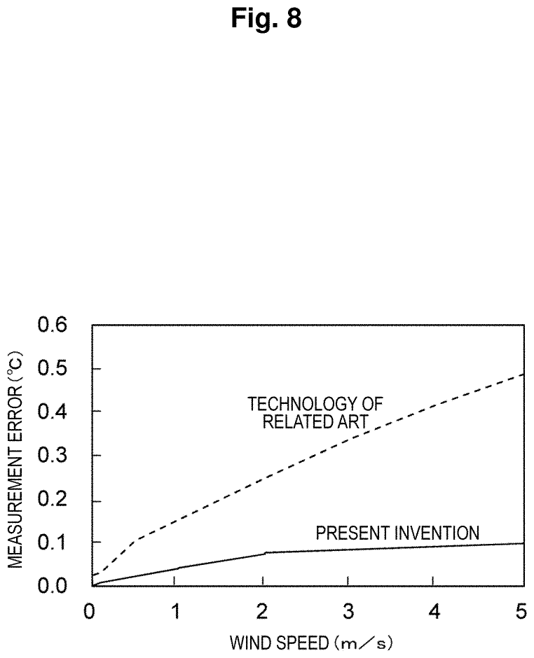

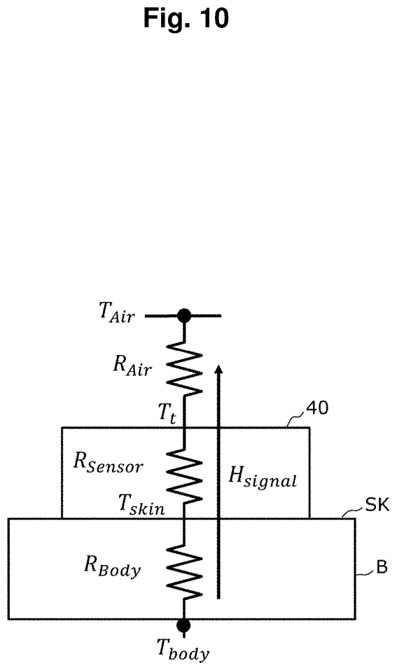

FIG. 8 illustrates a measurement result of a measurement error of the core temperature in the measurement device 1. FIG. 8 illustrates a relationship between the wind speed and the measurement error when wind is applied to the measurement device 1. The disclosure in the drawing is a measurement result in the configuration of FIG. 1, and the technique of the related art is a measurement result in the configurations of FIGS. 10 and 11. It is assumed that the wind applied to the measurement device 1 is a wind speed of 5 m/s at the maximum and jogging is performed at approximately 18 km/h. According to the measurement device of the present embodiment, it can be confirmed that the measurement error of the core temperature can be suppressed to 0.1° C. or less.

<Effects of Present Embodiment>

According to the present embodiment, the first member 10 including the measuring instrument that measures the heat flux and the second member 20 forming the air layer between the first member 10 and the second member 20 are provided, and the third member that transports the heat flux from the measurement target outside the first member to the upper surface portion of the second member is further provided between the first member and the second member. Therefore, even when the temperature outside the measuring instrument increases due to the heat flux transported to the upper surface portion of the second member and wind is generated around the measurement device, it is possible to suppress the change in the thermal resistance between the sensor and the outdoor air, suppress the leakage heat flux that causes the measurement error, and reduce the Leak Ratio to reduce the measurement error when measuring the core temperature.

<Configuration Example of Measurement Device>

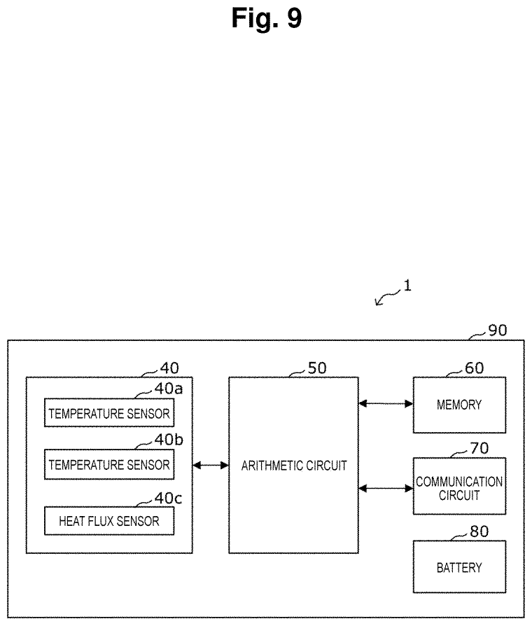

A configuration of the measurement device 1 according to the present embodiment will be described with reference to FIG. 9. As illustrated in FIG. 9, the measurement device 1 includes the configuration of the measurement device 1 described in FIG. 1, an arithmetic circuit 50 that estimates a core temperature, a memory 60, a communication circuit 70, and a battery 80.

The measurement device 1 includes, for example, the measuring instrument 40, the arithmetic circuit 50, the memory 60, the communication circuit 70 that functions as an I/F circuit with the outside, and the battery 80 that supplies power to the arithmetic circuit 50, the communication circuit 70, and the like on a sheet-like base material 90.

In the configuration example of FIG. 1, the arithmetic circuit 50 calculates an estimated value of a core temperature Tc from the temperatures Tskin and Tt measured by the temperature sensors 40a and 40b included in the measuring instrument 40 using Equation (i).

In the configuration example of FIG. 2, the arithmetic circuit 50 calculates an estimated value of the core temperature Tc using Equation (i) from the heat flux Hsignal and the skin temperature Tskin measured by the heat flux sensor 40c and the temperature sensor 40a, respectively, included in the measuring instrument 40.

The memory 60 stores the information on the one-dimensional biological heat transfer model based on the above-described Equation (i) and the estimation result of the core temperature. The memory 60 can be realized by a predetermined storage area in a rewritable nonvolatile storage device (for example, a flash memory or the like) provided in the measurement system.

The communication circuit 70 outputs the time-series data of the core temperature Tc of the living body B generated by the arithmetic circuit 50 to the outside. As the communication circuit 70, when data or the like is output by wire, an output circuit to which a USB or other cables can be connected is used, but for example, a wireless communication circuit conforming to Bluetooth (registered trademark), Bluetooth Low Energy, or the like may be used.

The sheet-like base material 90 functions as a base for placing the measurement device 1 including the measuring instrument 40, the arithmetic circuit 50, the memory 60, the communication circuit 70, and the battery 80, and further includes wiring (not illustrated) for electrically connecting these elements. Assuming that the measurement device 1 is connected on the skin of a living body, it is desirable to use a deformable flexible board for the sheet-like base material 90.

In addition, an opening is provided at a part of the sheet-like base material 90, and the temperature sensor 40a and the heat flux sensor 40c included in the measuring instrument 40 are placed on the base material 90 to be in contact with the measurement surface of the skin SK of the living body B from the opening.

Here, the measurement device 1 is realized by a computer. Specifically, the arithmetic circuit 50 is realized by, for example, a processor such as a CPU or a DSP executing various data processing according to a program stored in a storage device, such as a ROM, a RAM, and a flash memory, including a memory 60 provided in the measurement device 1. The program for causing the computer to function as the measurement device 1 can be recorded on a recording medium or provided through a network.

In FIG. 9, the measurement device 1 including the measuring instrument 40 described in FIG. 1 is configured integrally with another configuration including the arithmetic circuit 50, but the configuration of FIG. 1 may be configured separately from the arithmetic circuit 50, the memory 60, the communication circuit 70, and the battery 80. For example, the measurement device 1 and other configurations such as the arithmetic circuit 50 may be connected via wiring (not illustrated).

<Modification of Embodiment>

Although the embodiments of the measurement device of the present invention have been described above, the present invention is not limited to the described embodiments, and various modifications that can be assumed by those skilled in the art can be made within the scope of the invention described in the claims.

### REFERENCE SIGNS LIST

1 Measurement device
10 First member
20 Second member
30 Third member
31 Hole portion
40 Measuring instrument
40a, 40b Temperature sensor
40c Heat flux sensor
50 Arithmetic circuit
60 Memory
70 Communication circuit
80 Battery
90 Base material.

1-7. (canceled)

8. A measurement device comprising:
a measuring instrument configured to measure a heat flux transported from a measurement target;
a first member having a hollow structure and including the measuring instrument therein;
a second member having a hollow structure and covering the first member to define an air layer between the first member and the second member; and
a third member between the first member and the second member, the third member being configured to transport the heat flux from the measurement target outside the first member to an upper portion of the second member.

9. The measurement device according to claim 8, wherein:
the third member has a conical shape, and an upper portion of the conical shape of the third member is configured to be in contact with the upper portion of the second member at an inner surface of the second member.

10. The measurement device according to claim 9, wherein:
the third member has a frustum shape, and an upper surface portion of the frustum shape of the third member is configured to be in contact with the upper portion of the second member at the inner surface of the second member.

11. The measurement device according to claim 10, wherein:
the second member has a cylindrical shape; and
the third member has a truncated shape, and the upper surface portion of the truncated shape of the third member is configured to be in contact with the upper surface portion of the cylindrical shape of the second member from the inner surface of the second member.

12. The measurement device according to claim 11, wherein:
the third member includes a hole portion penetrating the upper surface portion of the truncated shape.

13. The measurement device according to claim 8, wherein:
the measuring instrument includes a first temperature sensor disposed on a measurement surface of the measurement target, and a second temperature sensor disposed inside the first member and facing the first temperature sensor.

14. The measurement device according to claim 8, wherein:
the measuring instrument includes a temperature sensor and a heat flux sensor which are disposed on a measurement surface of the measurement target.

15. A method comprising:
transporting a heat flux from a measurement target to a measuring instrument of a measurement device; and
measuring, by the measuring instrument, the heat flux, wherein the measurement device comprises:
a first member having a hollow structure and including the measuring instrument therein;
a second member having a hollow structure and covering the first member to define an air layer between the first member and the second member; and
a third member between the first member and the second member, the third member being configured to transport the heat flux from the measurement target outside the first member to an upper portion of the second member.

16. The method according to claim 15, wherein:
the third member has a conical shape, and an upper portion of the conical shape of the third member is configured to be in contact with the upper portion of the second member at an inner surface of the second member.

17. The method according to claim 16, wherein:
the third member has a frustum shape, and an upper surface portion of the frustum shape of the third member is configured to be in contact with the upper portion of the second member at the inner surface of the second member.

18. The method according to claim 17, wherein:
the second member has a cylindrical shape; and
the third member has a truncated shape, and the upper surface portion of the truncated shape of the third member is configured to be in contact with the upper surface portion of the cylindrical shape of the second member from the inner surface of the second member.

19. The method according to claim 18, wherein:
the third member includes a hole portion penetrating the upper surface portion of the truncated shape.

20. The method according to claim 15, wherein:
the measuring instrument includes a first temperature sensor disposed on a measurement surface of the measurement target, and a second temperature sensor disposed inside the first member and facing the first temperature sensor.

21. The method according to claim 15, wherein:
the measuring instrument includes a temperature sensor and a heat flux sensor which are disposed on a measurement surface of the measurement target.
